(19) United States
(12) Patent Application Publication
(10) Pub. No.: US 2005/0137380 A1
(43) Pub. Date: Jun. 23, 2005

(54) Processes for preparing benzoquinones and hydroquinones

(76) Inventors: Rathinam Jothi Mahalingam, Bangalore (IN); Sunil Ashtekar, Bangalore (IN); Jegadeesh Thampi, Bangalore (IN); Pramod Shankar Kumbhar, Bangalore (IN)

Correspondence Address: CANTOR COLBURN LLP, 55 GRIFFIN RD SOUTH, BLOOMFIELD, CT, 06002, US

(21) Appl. No.: 10/757,921

(22) Filed: Jan. 14, 2004

Related U.S. Application Data
(60) Provisional application No. 60/530,562, filed on Dec. 18, 2003.

(51) Int. Cl. C08G 64/00; C07C 50/04; C07C 37/06

(52) U.S. Cl. 528/196; 552/296; 568/772

(57) ABSTRACT

A process for the preparation of benzoquinones and hydroquinones includes oxidizing an aromatic hydroxy compound with oxygen or an oxygen containing gas, a copper containing catalyst, and a promoter to form the benzoquonone. A reduction reaction is employed to form the hydroquinone.

### CROSS-REFERENCE TO RELATED APPLICATIONS

This application claims priority to U.S. Provisional Application Ser. No. 60/530,562, filed Dec. 18, 2003.

### BACKGROUND

This disclosure generally relates to a process for preparing hydroquinone compounds from aromatic hydroxy compounds.

Hydroquinone compounds find applications in a wide range of industries including, among others, the polymer industry, the dye industry, the photographic industry and in medical applications. They are also known for fabricating polycarbonates for use in liquid crystal displays.

Prior methods for the preparation of hydroquinone compounds generally include oxidation of aromatic hydroxy compound to the corresponding benzoquinone compound followed by reduction of the benzoquinone to give the corresponding hydroquinone compound. The oxidation of aromatic hydroxy compounds to benzoquinones has been widely studied and some of the earlier methods for oxidation typically include oxidation of aromatic hydroxy compounds with an inorganic oxidizing agent like potassium permanganate, manganese dioxide and lead oxide or with oxygen in the presence of a catalyst. However, the disadvantage associated with this method is the need for stoichiometric amounts of expensive oxidizing agents and the necessity to treat or regenerate the metals in a reduced valency state.

Other techniques employed in the preparation of substituted and unsubstituted benzoquinones include the use of catalyst systems containing copper. Aqueous solution having cupric chloride and lithium chloride in 30-50% concentration (molar ratio phenol:cupric chloride dihydrate: lithium chloride:: 1:1:4) have been used to effect the oxidation of 2,3,6-trimethyl phenol to 2,3,5-trimethylbenzoquinone. However, use of such high quantities of catalyst leads to formation of undesired chloro-substituted compounds.

Copper containing catalysts in the presence of promoters like thiocyanate, cyanate, cyanide ions or halogens in water and water miscible solvents have also been disclosed for the oxidation of phenols and alkyl substituted phenols to their corresponding benzoquinones. However, oxygen pressures employed are relatively high and the promoters are known toxic agents.

Use of high concentrations of about 20-80% of copper halogen complex M₁[Cu(II)ₘXₙ] combined with either an alkali metal halide or with an alkali metal halide and a cupric hydroxide and/or cuprous chloride in a medium of water and an aliphatic alcohol containing about 5 to about 10 carbon atoms to prepare 2,3,5-trimethylbenzoquinone from 2,3,6-trimethyl phenol are also disclosed. However, the molar ratio of copper halogen complex to phenol substrate is 0.1-5:1, which leads to substantial formation of chloro compounds. One-pot oxidation processes to obtain various hydroquinones and substituted hydroquinones from phenol or substituted phenol are also disclosed, in that a divalent copper catalyst promoted with an alkali metal hydroxide or a monovalent copper catalyst promoted with water is used in the presence of oxygen, followed by hydrogenation with hydrogen gas at an elevated pressure and temperature in the same system after flushing out the oxygen gas. The quantity of oxygen and hydrogen used here is relatively high.

Accordingly, there is a need in the art for a commercial and cost effective process for manufacturing hydroquinones with high conversion and high selectivity.

### BRIEF SUMMARY

Disclosed herein is a process for preparing hydroquinone compounds, the process comprising oxidizing an aromatic hydroxy compound in a solvent with an oxygen gas or an oxygen-containing gas mixture in the presence of a catalytic amount of a copper containing catalyst and a promoter to form the benzoquinone compound, wherein said copper containing catalyst comprises a mixture of a halide salt and a copper salt, or a double salt of the halide salt and the copper salt, and wherein the catalytic amount of the copper containing catalyst is less than or equal to 0.1 mole per mole of aromatic hydroxy compound. The process may further comprise reducing the so-formed benzoquinone compounds to the corresponding hydroquinone compounds.

In another embodiment, a process for preparing 2-methylhydroquinone, comprises oxidizing ortho-cresol with oxygen gas in a solvent selected from the group consisting of methylisobutylketone and methylethylketone in the presence of lithium trichlorocuprate dihydrate catalyst, and N-methylpyrrolidone promoter at a pH of about 1 to about 5 to form 2-methyl benzoquinone, wherein the lithium trichlorocuprate dihydrate catalyst is less than or equal to 0.1 mole per mole of the ortho-cresol; reducing the 2-methyl benzoquinone; and isolating the 2-methylhydroquinone.

In yet another embodiment, process for preparing a benzoquinone compound comprises: oxidizing an aromatic hydroxy compound in a solvent with an oxygen gas or an oxygen-containing gas mixture in the presence of a copper containing catalyst and a promoter to form the benzoquinone compound, wherein said promoter comprises an organic amide.

The present disclosure may be understood more readily by reference to the following detailed description of the various features of the disclosure and the examples included therein.

### DETAILED DESCRIPTION

Disclosed herein is a process for preparing benzoquinone compounds that is cost effective with high conversion and high selectivity. The benzoquinone compounds are prepared from aromatic hydroxy compounds preferably of the formula: 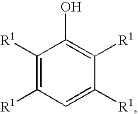
wherein R¹ is independently selected from the group consisting of a hydrogen and a hydrocarbyl group, wherein the hydrocarbyl group is selected from the group consisting of an alkyl group containing 1 to about 18 carbon atoms, an aryl group containing about 6 to about 20 carbon atoms, an aralkyl group containing about 6 to about 12 carbon atoms and an alkylaryl group containing about 7 to about 16 carbon atoms. The benzoquinone compounds are optionally reduced to provide the corresponding hydroquinone compounds. The hydroquinone compounds have a variety of applications, including use as monomer in the preparation of polycarbonates, as intermediate in the preparation of vitamins and dyestuffs, and in the photographic industry.

The process for the preparation of the benzoquinone compounds generally comprises oxidizing an aromatic hydroxy compound with oxygen gas or an oxygen-containing gas mixture in a solvent in the presence of a catalytic amount of a copper containing catalyst and a promoter. The pressure, temperature, and pH during the oxidation is effective to form a benzoquinone compound. The so-formed benzoquinone compound could then be optionally reduced with a reducing agent to provide the corresponding hydroquinone compound. The hydroquinone compound may then be isolated using an anti-solvent.

The singular forms “a”, “an” and “the” include plural referents unless the context clearly dictates otherwise. “Optional” or “optionally” means that the subsequently described event or circumstance may or may not occur, and that the description includes instances where the event occurs and instances where it does not.

Unless otherwise specified, the term “alkyl” as used herein is intended to designate straight chain alkyls and branched chain alkyl groups. Illustrative non-limiting examples of suitable straight chain and branched chain alkyl groups include methyl, ethyl, n-propyl, isopropyl, n-butyl, sec-butyl, tertiary-butyl, pentyl, neopentyl, hexyl, heptyl, octyl, nonyl, decyl, undecyl and dodecyl. Suitable aralkyl groups include, but are not intended to be limited to, benzyl, phenylbutyl, phenylpropyl, and phenylethyl. In various other embodiments, the term “aryl” or “aromatic” groups are intended to designate monocyclic or polycyclic moieties containing about 6 to about 20 ring carbon atoms. Some illustrative non-limiting examples of these aromatic groups include phenyl, biphenyl, and naphthyl.

The aromatic hydroxy compound is preferably oxidized directly to the corresponding benzoquinone in a solvent in the presence of a copper containing catalyst and a promoter at a pH of about 1 to about 5. The oxidation is carried out using oxygen gas or an oxygen-containing gas mixture comprising nitrogen, ambient air, helium or argon. More specifically, the oxidation is carried out using oxygen gas. The copper containing catalyst comprises a mixture of a halide salt and a copper salt, or a double salt of a halide salt and a copper salt. The promoter is particularly an organic amide. Alternatively, a copper salt alone can be used as a catalyst in combination with the organic amide promoter. During oxidation, the pH is maintained at about 1 to about 5 by the addition of an acid, if required. The so-formed benzoquinone may be subsequently reduced with a reducing agent to give the hydroquinone compound.

Specific examples of suitable aromatic hydroxy compounds include, but are not intended to be limited to, 2,6-dimethylphenol, 2,3,6-trimethylphenol, 2,6-di-tert-butylphenol, 2-tert-butylphenol, alpha-naphthol, meta-cresol, ortho-cresol, ortho-phenylphenol, ortho-chlorophenol, ortho-benzylphenol, 2,6-dichlorophenol, ortho-vinylphenol, and mixtures of the foregoing aromatic hydroxy compounds. In one particular embodiment, the aromatic hydroxy compound is ortho-cresol.

Specific examples of the halide salt include halide salts of the formula, M-X, wherein M comprises an alkali metal, an ammonium ion, or an organoammonium ion, and X is selected from the group consisting of chloride, bromide, and iodide. The organoammonium ion is specifically of the formula, R₂—[NH₃]⁺, wherein R² is a monovalent hydrocarbyl group containing 1 to about 6 carbon atoms, and wherein the hydrocarbyl group includes, but is not limited, to isopropyl, n-butyl, tertiary butyl, and isopentyl groups.

Suitable halide salts include, but are not intended to be limited to, sodium chloride, lithium chloride, potassium chloride, cesium chloride, sodium bromide, ammonium bromide, potassium bromide, cesium bromide, sodium iodide, lithium iodide, potassium iodide, cesium iodide, isopropyl ammonium bromide, and mixtures of the foregoing halide salts. In one particular embodiment, the halide salt is lithium chloride.

Specific examples of copper salts include, a cuprous salt, a cupric salt, or mixtures of the foregoing copper salts. More specifically the copper salts include, but are not limited to, cuprous chloride, cuprous bromide, cuprous iodide, cupric chloride, cupric bromide, cupric iodide, cuprous acetate, and cupric acetate. More particularly the copper salt is cupric chloride.

Specific examples of a double salt of the halide salt and the copper salt include a compound of the formula, M[CuX₃], or a compound of the formula, M₂[CuX4]; wherein M is selected from the group consisting of an alkali metal, an ammonium ion, and an organoammonium ion; and X is selected from the group consisting of chloride, bromide, and iodide. Alkali metals include, but are not limited to sodium, potassium, lithium, and cesium. The organoammonium ion is of the formula, R²-[NH₃]⁺; wherein R² is a monovalent hydrocarbyl group containing 1 to about 6 carbon atoms; wherein the hydrocarbyl group is selected from a group including isopropyl, isobutyl, butyl, tertiary butyl and isopentyl groups.

More specifically the double salts include, but are not intended to be limited to, lithium trichlorocuprate dihydrate, ammonium trichlorocuprate dihydrate, diammonium tetrachlorocuprate dihydrate, dipotassium tetrachlorocuprate dihydrate, cesium trichlorocuprate dihydrate, dicesium tetrachlorocuprate dihydrate, dilithium tetrabromocuprate hexahydrate, potassium tribromocuprate, diammonium tetrabromocuprate dihydrate, and cesium tribromocuprate. In one particular embodiment, the double salt is lithium trichlorocuprate dihydrate.

The double salts can be prepared by known procedures, for example by following the methods described in Mellor's Comprehensive Treatment on Inorganic and Theoretical Chemistry, 1963, Vol. III, pages 182-191 (Longman).

The amount of the copper containing catalyst employed, when a double salt is used, may preferably comprise less than or equal to 0.1 moles per mole of aromatic compound, with 0.01 moles to 0.1 mole per mole of aromatic hydroxy compound more preferred, and with about 0.025 moles to about 0.075 moles per mole of aromatic hydroxy compound even more preferred. Likewise, when a combination of the halide salt and the copper salt is used, the ratio of the catalyst to the aromatic hydroxy compound may preferably comprise less than 0.1 moles per mole of aromatic compound, with 0.01 moles to 0.1 mole per mole of aromatic hydroxy compound more preferred, and with about 0.025 moles to about 0.075 moles per mole of aromatic hydroxy compound even more preferred, wherein the catalyst moles consist of equimolar quantities of the halide salt and the copper salt.

Specific examples of promoters include organic amides. More specifically, the organic amides include, but are not intended to be limited to, N-methyl-2-pyrrolidone, N,N-dimethylformamide, N,N-diphenylformamide, N-cyclohexyl-N-methylformamide, N-methyl-acetamide and N-phenyl-N-methylformamide. The weight ratio of the promoter to the aromatic hydroxy compound is about 0.05 to about 0.7. In one particular embodiment, the weight ratio of promoter to aromatic hydroxy compound is about 0.25 to about 0.5.

Advantageously, the use of the promoter reduces the amount of catalyst employed in the reduction of aromatic hydroxy compounds to corresponding benzoquinones, thereby leading to a significant reduction in the formation of undesired chloro compounds. The lower amounts of catalyst result in increased conversions and higher selectivities.

The oxidation of the aromatic hydroxy compound with the oxidizing agent is in general carried out in a partially water-miscible solvent comprising an organic ketone. The partially water-miscible solvent is preferably an organic ketone containing about 4 to about 10 carbon atoms. Exemplary partially water-miscible solvents include, but are not limited to, methyl isobutyl ketone and methyl ethyl ketone. Alternatively, a water-miscible solvent comprising an organic alcohol, an organic sulfoxide, organic ether, or combinations of the foregoing water-miscible solvents, is employed for the oxidation of the aromatic hydroxy compound with the oxidizing agent. The water-miscible solvent is preferably selected from the group consisting of an alcohol containing 1 to about 8 carbon atoms, a sulfoxide containing about 2 to about 4 carbon atoms, and an ether containing about 4 to about 12 carbon atoms. Exemplary water-miscible solvents include, but are not limited to isopropyl alcohol, octanol, dimethylsulfoxide and monoethylene glycol dimethyl ether (monoglyme).

The oxidation of the aromatic hydroxy compound with the oxidizing agent may be specifically carried out at a pH of 1 to about 5, and more particularly, at a pH of about 2. The required pH is maintained by optionally adding an acid. The acids that are optionally used to maintain the pH include, but are not limited to, a water-miscible organic acid, a water-miscible inorganic acid, or combinations of the foregoing acids. The organic acid is preferably an aliphatic carboxylic acid. Suitable aliphatic organic acids include, but are not limited to acetic acid, formic acid, oxalic acid, propionic acid, butanoic acid and combinations of the foregoing acids. The inorganic acid is preferably a protic acid selected from the group of hydrochloric acid, sulfuric acid and combinations of the foregoing acids. Since acid is used optionally, the amount of acid employed in the reaction is sufficient enough for adjusting and maintaining the pH from 1 to about 5, more particularly at a pH of about 2.

The oxidation of the aromatic hydroxy compound with the oxidizing agent may be specifically carried out at a temperature of about 20° C. to about 75° C., and more particularly, at a temperature of about 50° C. to about 60° C. The pressure during the reaction is at about 17.5 Newtons per square centimeter (N/cm²) to about 172.4 N/cm², and more particularly, at a pressure of about 52.5 N/cm² to about 137.0 N/cm². The time required for the oxidation may vary about 5 hours to about 24 hours, and more particularly, about 5 hours to about 10 hours.

The oxidation of the aromatic hydroxy compound in presence of the catalyst and the promoter takes place in a pressurized reactor vessel. Laboratory scale experiments may be carried out in Parr pressurized reactor vessel commercially available from the Parr Instrument Company. The workup of the reaction mixture differs depending on whether the solvent used is partially water-miscible or water-miscible. When partially water-miscible solvent is used, the reaction mixture after oxidation step is subjected to a water wash to wash out the halide and copper salt or the double salt. Optionally, the organic layer including the partially water-miscible solvent containing the benzoquinone is used for the reduction step. When water-miscible solvent is used, the reaction mixture after oxidation is diluted with water and then extracted in a solvent such as toluene or xylene, the solvent is evaporated out to obtain a residue. Optionally, the residue is taken in a solvent suitable for reduction. The solvents for reduction can be selected from alcohols containing 1 to about 4 carbon atoms, acetic acid, and water. Suitable alcohols include, but are not intended to be limited to, methanol, ethanol, isopropyl alcohol, ethylene glycol, and mixtures of the foregoing alcohols.

Suitable reducing agents comprise hydrogen gas or hydrogen gas containing mixtures in presence of a reduction catalyst or the reducing agent. Suitable reducing agents are selected from the group consisting of sodium borohydride, sodium dithionate, lithium aluminum hydride, sodium hydrosulfite and sodium bisulfite. Suitable reduction catalysts are selected from the group including Raney nickel, palladium-carbon, palladium supported on alumina, palladium supported on silica, platinum supported on charcoal, platinum supported on alumina, platinum supported on silica, platinum supported on silica-alumina, palladium supported on silica-alumina. tin, iron-hydrochloric acid, and zinc-acetic acid. After completion of the reduction, the solvent is distilled off and an anti-solvent is added to the residue to precipitate the hydroquinone compound.

The anti-solvent employed in the isolation after the reduction step is selected from the group including, but not limited to, toluene, xylenes, chlorobenzenes, and heptane. In one particular embodiment, the anti-solvent is toluene.

As previously discussed, the hydroquinone compounds find various end use applications in the polymer, dyestuff, pharmaceutical, photographic industries and in medical applications. Polycarbonates particularly containing methyl hydroquinone units are known to exhibit liquid crystalline properties. Suitable methods for preparation of these polycarbonates include melt-transesterification reaction of diphenylcarbonate and mixtures of methyl hydroquinone and bisphenol; and melt polymerization methods in presence of quaternary phosphonium salts, sodium hydroxide or tetraalkylammonium salts as catalyst systems. The hydroquinone compounds could also be used to prepare polyesters when coupled with other monomers by melt polymerization techniques as is known in the art.

The disclosure is explained in more detail with reference to the following non-limiting Examples, which are only illustrative, but not limitative.

### EXAMPLES

In the following examples and comparative examples, a high performance liquid chromatography (HPLC) method was used to quantify the conversion of an aromatic hydroxy compound to a benzoquinone compound. The HPLC was initially calibrated using standard Aldrich samples of aromatic hydroxy compound and corresponding benzoquinone and hydroquinone. The standard samples were diluted with an internal standard solution of N-methyl benzamide in acetonitrile and a sample injected into a C-18 reverse phase column. Each reaction mixture was then diluted with an internal standard solution of N-methyl benzamide in acetonitrile and a sample injected into a C-18 reverse phase column. Samples at specific time intervals were analyzed and compared to the HPLC chromatogram of the standard sample to determine the conversion of aromatic hydroxy compound and selectivity towards corresponding benzoquinone after oxidation and hydroquinone after reduction. The pH is measured using a glass membrane electrode that is calibrated using 2 point calibration with standard buffers. The accuracy of the pH meter is ±0.01.

### Example 1

In this example, a lithium trichloro cuprate dihydrate catalyst stock solution was prepared. A mixture of cupric chloride (40.02 gm) and lithium chloride (9.97 gm) were placed in a 100 milliliter (ml) volumetric flask and the volume made up to 100 ml with water.

### Example 2

In this example, 2-methyl hydroquinone was prepared. A mixture of ortho-cresol (127 grams (g)), lithium trichoro cuprate dihydrate solution (22.3 milliliters) as prepared in Example 1, N-methylpyrrolidone (41.81 g), acetic acid (41.81 g) and methyl isobutyl ketone (842.64 g) was charged to a 3.7 liter Parr vessel. The pressure vessel was closed and pressurized with oxygen to 35 N/cm² and depressurized to atmospheric pressure. This was repeated thrice. The reactor was heated to 50° C. and pressurized with oxygen to a pressure of 70 N/cm². This pressure was maintained throughout the experiment by replenishment with oxygen as needed. The reaction was monitored by high-performance liquid chromatography (HPLC) for the conversion of ortho-cresol and the selectivity of conversion to methyl benzoquinone. After about 24 hours, the conversion of ortho-cresol was 74.7% with a selectivity of conversion to methyl-benzoquinone of 74.5%. The reactor was cooled to room temperature (25° C.) and the reaction mixture was washed three times with water. The water quantity used each time for the washing was equal to about one-quarter the weight of the reaction mixture.

To effect reduction to the corresponding hydroquinone, a mixture of the above washed reaction mixture (200 g), Raney Nickel water slurry (3.6 g) (approx 50% wt/wt nickel to water) was charged to a 600 milliliter Parr pressure vessel and the pressure vessel was closed and pressurized with nitrogen to 35 N/cm² and depressurized to atmospheric pressure. This was repeated thrice. The reactor was heated to 80° C. and pressurized with hydrogen to a pressure of 63 N/cm². This pressure was maintained throughout the experiment by replenishment with hydrogen. The reaction was monitored by HPLC for the conversion of methyl benzoquinone and the selectivity of conversion to methyl hydroquinone. The conversion of methyl benzoquinone was quantitative with a selectivity of conversion towards methyl hydroquinone of 67% after about 5.5 hrs. The reactor was cooled and the reaction mixture was distilled to remove the methyl isobutyl ketone solvent. The residue was used for the isolation and purification of methyl hydroquinone. The weight of the residue obtained was about 34.59 g. HPLC analysis showed 23.4% methyl hydroquinone, 15.1% ortho-cresol.

To the above residue was added toluene (100 ml) and the resultant mixture was stirred for 12 hours at room temperature (25° C.). The precipitated methyl hydroquinone was filtered and the mother liquor cooled to about 10° C. to recover second crop of methyl hydroquinone. Total crude product obtained was 8 g and it was washed with 4 volumes of toluene and dried.

The yield of the purified product obtained was 7.7 gm. The purity of the product was 99% as analyzed by HPLC.

### Example 3

In this example, 2-methyl hydroquinone was prepared by following the same procedure as mentioned in Example 2 except that ortho-cresol (61.97 grams), lithium trichoro cuprate dihydrate solution (11.99 g), N,N-dimethylformamide (20.88 g), acetic acid (11.27 g) and methyl isobutyl ketone (419.12 g) were used. After about 24 hours, the conversion of ortho-cresol was 77% with a selectivity of conversion towards methyl-benzoquinone of 83%. Reduction of the methyl benzoquinone reaction mixture was carried out in the same manner as described in Example 2, except in this case the entire reaction mixture was taken for reduction. The conversion of methyl benzoquinone was quantitative with a selectivity of conversion towards methyl hydroquinone of 62%. After about 5.5 hrs, the product was isolated in the same manner as in Example 2. The yield of pure product obtained was 16.46 grams.

### Examples 4-7

In these examples, 2-methyl benzoquinone was prepared using the components set forth in Table 1 and the reaction parameters set forth in Table 2.

The general procedure followed in these reactions includes charging a mixture of the aromatic hydroxy compound, catalyst, promoter, acid, and solvent in a 450 milliliter Parr pressure vessel under a continuous flow of oxygen at 300 milliliter per hour. The reactor is heated and pressurized with oxygen to a pressure of 70 N/cm². An acidic pH is maintained by addition of acetic acid. The reaction is monitored by HPLC for the conversion of ortho-cresol and the selectivity of conversion towards methyl benzoquinone. These reactions were carried out in methyl isobutyl ketone to study the effect of temperature on the conversion and selectivity in the oxidation of ortho-cresol to 2-methyl benzoquinone. The results of the examples are shown in Table 2.

TABLE 1

| | | | | | Catalyst |
|---|---|---|---|---|---|
| | | | | | Lithium |
| | | | | | trichloro |
| | Aromatic | | | | cuprate |
| | hydroxy | Solvent | Promoter | Acid | dihydrate |
| | phenol | methyl | N-methyl | acetic | aqueous |
| Ex. | ortho-cresol | isobutyl ketone | pyrrolidone | acid | solution |
| No. | (g) | (g) | (g) | (g) | (g) |
| 4 | 16.95 | 79.0 | 4.04 | 4.08 | 2.95 |
| 5 | 12.78 | 82.6 | 4.24 | 4.27 | 2.22 |
| 6 | 14.38 | 81.4 | 4.08 | 4.17 | 2.84 |
| 7 | 14.90 | 81.4 | 5.13 | 5.26 | 4.01 |

1 methyl ethyl ketone

TABLE 2

| | | | | | Methyl |
|---|---|---|---|---|---|
| Example | Temperature | | Time | o-cresol % | benzoquinone |
| No. | (° C.) | pH | (hours) | conversion | % selectivity |
| 4 | 42 | 2.1 | 8 | 35.5 | 82.3 |
| 5 | 50 | 2.1 | 12 | 57.2 | 90.0 |
| 6 | 55 | 1.9 | 8 | 81.0 | 68.7 |
| 7 | 60 | 1.5 | 5 | 100.0 | 67.4 |

The results indicate increasing the reaction temperature results in an increased conversion. However, it is noted that selectivity is higher at the lower processing temperatures and appears to decrease with increasing temperature.

### Examples 8-9

In these examples, 2-methyl benzoquinone was prepared using the components set forth in Table 3 and the reaction parameters set forth in Table 4 following the general procedure followed in Examples 4-7. The results, as shown in Table 4, illustrate the effect of catalyst on the conversion and selectivity in the formation of 2-methyl-benzoquinone from ortho cresol.

TABLE 3

| | | | | | Catalyst |
|---|---|---|---|---|---|
| | | | | | Lithium |
| | | | | | trichloro |
| | Aromatic | | | | cuprate |
| | hydroxy | Solvent | Promoter | Acid | dihydrate |
| | phenol | methyl | N-methyl | acetic | aqueous |
| Ex. | ortho-cresol | isobutyl ketone | pyrrolidone | acid | solution |
| No. | (g) | (g) | (g) | (g) | (g) |
| 8 | 14.38 | 80.9 | 4.08 | — | 0.83 |
| 9 | 12.86 | 83.2 | 4.16 | 4.17 | 4.44 |

TABLE 4

| | | | | | Methyl |
|---|---|---|---|---|---|
| Example | Temperature | | Time | o-cresol % | benzoquinone |
| No. | (° C.) | pH | (hours) | conversion | % selectivity |
| 8 | 60 | 2.1 | 5 | 24.8 | 56.4 |
| 9 | 50 | 3.1 | 2 | 28.3 | 65.2 |

### Examples 10-11

In these examples, benzoquinones were prepared using the components set forth in Table 5 and the reaction parameters set forth in Table 6 following the general procedure followed in Examples 4-7. The results, as shown in Table 6, illustrate the effect of reaction conditions on phenol and 2,6-dimethylphenol.

TABLE 5

| | | | | | Catalyst |
|---|---|---|---|---|---|
| | | | | | Lithium |
| | | | | | trichloro |
| | Aromatic | | | | cuprate |
| | hydroxy | Solvent | Promoter | Acid | dihydrate |
| | phenol | methyl | N-methyl | acetic | aqueous |
| Ex. | ortho-cresol | isobutyl ketone | pyrrolidone | acid | solution |
| No. | (g) | (g) | (g) | (g) | (g) |
| 10 | 13.040¹ | 79.4 | 4.880 | 4.990 | 2.00 |
| 11 | 17.27² | 80.2 | 4.890 | 5.020 | 2.00 |

¹phenol
²2,6-dimethyl xylenol

TABLE 6

| | | | | | Methyl |
|---|---|---|---|---|---|
| Example | Temperature | | Time | o-cresol % | benzoquinone |
| No. | (° C.) | pH | (hours) | conversion | % selectivity |
| 10 | 60 | 1.8 | 6 | 15.9 | 100.0 |
| 11 | 60 | 2.1 | 6 | 100 | 89.0 |

The results show that the process can be employed with substituted as well as substituted aromatic hydroxy phenols. For example, as shown in Example 10, the aromatic hydroxy phenol is an unsubstituted phenol with selectivity of about 100%. It is expected that process optimization will increase percent conversion.

### Examples 12-20

In these examples, 2-methyl benzoquinone were prepared using the components set forth in Table 7 and the reaction parameters set forth in Table 8 following the general procedure followed in examples 4-7. The examples study the effect of water-miscible solvent, different catalyst, different promoter different acid, higher pressure and no acid on the formation of 2-methyl benzoquinone from ortho-cresol. The conversion and selectivity results are tabulated in Table 8.

TABLE 7

| | | | | | Catalyst |
|---|---|---|---|---|---|
| | | | | | Lithium |
| | | | | | trichloro |
| | Aromatic | Solvent | | | cuprate |
| | hydroxy | methyl | Promoter | | dihydrate |
| | phenol | isobutyl | N-methyl | Acid | aqueous |
| Ex. | ortho-cresol | ketone | pyrrolidone | acetic acid | solution |
| No. | (g) | (g) | (g) | (g) | (g) |
| 12 | 12.82 | 83.5³ | 4.2 | 4.23 | 2.22 |
| 13 | 24.89 | 168.9 | 4.89 | 5.02 | 3.24⁴ |
| 14 | 12.22 | 83.2 | 4.31⁵ | 4.27 | 2.12 |
| 15⁶ | 25.64 | 165.8 | 8.32 | 8.31⁷ | 2.78 |
| 16 | 10.49 | 98.0⁸ | 5.14 | 5.21 | 1.61 |
| 17 | 12.7 | 83.4 | 4.28 | 4.14 | 2.32⁹ |
| 18 | 15.1 | 82.0 | 5.21 | 5.24 | 1.49¹⁰ |
| 19¹¹ | 14.49 | 82.6 | 5.14 | 5.21 | 5.08 |
| 20¹² | 15.04 | 85.2 | 5.24 | — | 2.01 |

³monoglyme
⁴isorpropylammonium trichloro cuprate dihydrate
⁵N,N-dimethylformamide
⁶oxygen pressure 137 N/cm²
⁷propionic acid
⁸methyl ethyl ketone
⁹cupric chloride dihydrate solution
¹⁰The amount of catalyst corresponds to cupric chloride dihydrate (1.1985 grams) and lithium chloride (0.2977 grams)
¹¹oxygen pressure 105 N/cm²
¹²no acid used

TABLE 8

| | | | | | Methyl |
|---|---|---|---|---|---|
| Example | Temperature | | Time | o-cresol % | benzoquinone |
| No. | (° C.) | pH | (hours) | conversion | % selectivity |
| 12 | 55 | 3.1 | 10 | 75.2 | 86.2 |
| 13 | 55 | 3.7 | 5 | 28.3 | 65.2 |
| 14 | 50 | 2.1 | 6 | 48.9 | 92.7 |
| 15 | 55 | 3.5 | 9 | 97.4 | 99.5 |
| 16 | 60 | 2.1 | 5 | 54.4 | 71.3 |
| 17 | 50 | 3.0 | 6 | 26.9 | 53.3 |
| 18 | 55 | 1.7 | 7 | 79.9 | 54.7 |
| 19 | 55 | 2.1 | 4 | 100.0 | 65.8 |
| 20 | 60 | 3.2 | 4 | 41.7 | 100.0 |

The results demonstrate the robustness of the oxidation process for oxidizing the aromatic hydroxy phenols to give the corresponding benzoquinones.

### Comparative Examples 1-2

In these examples, 2-methyl benzoquinone was prepared using the components set forth in Table 9 and the reaction parameters set forth in Table 10 following the general procedure of examples 4-7. The conversions and selectivities are included in Table 10 below. The oxidation processes of comparative Examples 1 and 2 were without the promoter.

TABLE 9

| | | | | | Catalyst |
|---|---|---|---|---|---|
| | | | | | Lithium |
| | | | | | trichloro |
| | | Solvent | | | cuprate |
| | Substrate | Methyl | Promoter | Acid | dihydrate |
| | ortho- | isobutyl | N-methyl | Acetic | aqueous |
| Comparative | cresol | ketone | pyrrolidone | acid | solution |
| Examples | (g) | (g) | (g) | (g) | (g) |
| 1 | 10.83 | 137¹ | — | — | 1.63 |
| 2 | 20.36 | 100² | — | — | 1.06 |

¹methyl ethyl ketone
²isopropyl alcohol

TABLE 10

| | | | | O-cresol | Methyl |
|---|---|---|---|---|---|
| Comparative | Temperature | | Time | (% | benzoquinone |
| Examples | (° C.) | pH | (hours) | Conversion) | (% selectivity) |
| 1 | 60 | 2.1 | 5 | 15.88 | 100.00 |
| 2 | 70 | 1.6 | 17 | 19.94 | 86.17 |

The results show that in absence of a promoter, the selectivities are relatively high but the conversions are very low.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

1. A process for preparing a benzoquinone compound, said process comprising:
oxidizing an aromatic hydroxy compound in a solvent with an oxygen gas or an oxygen-containing gas mixture in the presence of a catalytic amount of a copper containing catalyst and a promoter to form the benzoquinone compound, wherein said copper containing catalyst comprises a mixture of a halide salt and a copper salt, or a double salt of the halide salt and the copper salt, and wherein the catalytic amount of the a copper containing catalyst less than or equal to 0.1 mole per mole of aromatic hydroxy compound.

2. The process of claim 1, further comprising reducing said benzoquinone compound with a reducing agent to provide a hydroquinone compound.

3. The process of claim 1, wherein said aromatic hydroxy compound has a formula: 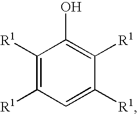
wherein R¹ is independently selected from the group consisting of a hydrogen and a hydrocarbyl group

4. The process of claim 1, wherein said aromatic hydroxy compound is selected from the group consisting of phenol, 2,6-dimethyl phenol, 2,6-di-tertiary-butyl phenol, 2-tertiary-butyl phenol, alpha-naphthol, meta-cresol, ortho-cresol, ortho-phenylphenol, ortho-benzylphenol, 2,3,6-trimethyl phenol, ortho-vinylphenol, 2-sopropylphenol, 2,6-diisopropylphenol, 2,3,5,6-tetramethylphenol, 2,3,5-trimethylphenol, and mixtures of the foregoing aromatic hydroxy compounds.

5. The process of claim 1, wherein said aromatic hydroxy compound is ortho-cresol.

6. The process of claim 1, wherein said oxygen-containing gas mixture comprises nitrogen, ambient air, helium, or argon.

7. The process of claim 1, wherein said halide salt is of the formula, M-X; wherein M comprises an alkali metal, an ammonium ion, or an organoammonium ion; and X is selected from the group consisting of chloride, bromide, and iodide.

8. The process of claim 7, wherein said organoammonium ion is of the formula, R²—[NH₃]⁺, wherein R² is a monovalent hydrocarbyl group containing 1 to about 6 carbon atoms.

9. The process of claim 1, wherein said copper salt comprises a cuprous salt, a cupric salt, or mixtures of the foregoing copper salts.

10. The process of claim 1, wherein said copper salt is selected from the group consisting of cuprous chloride, cuprous bromide, cuprous iodide, cupric chloride, cupric bromide, cupric iodide, cuprous acetate, and cupric acetate.

11. The process of claim 1, wherein said double salt of said halide salt and said copper salt comprises a compound of the formula, M[CuX₃], or a compound of the formula, M₂[CuX4]; wherein M is selected from the group consisting of an alkali metal, an ammonium ion, and an organoammonium ion; and X is selected from the group consisting of chloride, bromide, and iodide.

12. The process of claim 1, wherein said double salt of said halide salt and said copper salt is at least one selected from the group consisting of lithium trichlorocuprate dihydrate, ammonium trichlorocuprate dihydrate, diammonium tetrachlorocuprate dihydrate, dipotassium tetrachlorocuprate dihydrate, cesium trichlorocuprate dihydrate, dicesium tetrachlorocuprate dihydrate, dilithium tetrabromocuprate hexahydrate, potassium tribromocuprate, diammonium tetrabromocuprate dihydrate, and cesium tribromocuprate.

13. The process of claim 1, wherein said double salt of said halide salt and said copper salt is lithium trichlorocuprate dihydrate.

14. The process of claim 1, wherein oxidizing said aromatic hydroxy compound is carried out at a pH from 1 to about 5

15. The process of claim 1, further comprising adjusting a pH during oxidation to maintain said pH at about 1 to about 5.

16. The process of claim 1, further comprising adding an acid and maintaining a pH at about 1 to about 5.

17. The process of claim 1, wherein said promoter comprises an organic amide.

18. The process of claim 1, wherein said promoter is selected from the group consisting of N-methyl-2-pyrrolidone, N,N-dimethylformamide, N,N-diphenylformamide, N-methyl-acetamide, N-cyclohexyl-N-methylformamide, and N-phenyl-N-methylformamide.

19. The process of claim 1, wherein oxidizing the aromatic hydroxy compound is carried out at a temperature of about 20° C. to about 75° C.

20. The process of claim 1, wherein oxidizing the aromatic hydroxy compound is carried out under a pressure is about 17.5 Newtons per square centimeter to about 172.4 Newtons per square centimeter.

21. The process of claim 1, wherein oxidizing the aromatic hydroxy compound is carried out in presence of a water-miscible solvent.

22. The process of claim 1, wherein oxidizing the aromatic hydroxy compound is carried out in presence of a partially water-miscible solvent.

23. The process of claim 22, wherein said partially water-miscible solvent comprises an organic ketone solvent containing about 4 to about 10 carbon atoms or combinations of said organic ketone solvent.

24. The process of claim 21, wherein said water-miscible solvent comprises an organic alcohol containing 1 to about 8 carbon atoms, an organic sulfoxide containing about 2 to about 4 carbon atoms, an organic ether containing about 4 to about 12 carbon atoms, or combinations of the foregoing solvents.

25. The process of claim 21, wherein said water-miscible solvent is selected from the group consisting of Isopropyl alcohol, octanol, dimethylsulfoxide monoglyme, and combinations of the foregoing solvents.

26. The process of claim 22, wherein said partially water-miscible solvent is selected from the group consisting of methylisobutylketone, methylethylketone, and combinations of the foregoing solvents.

27. The process of claim 1, wherein said promoter and aromatic hydroxy compound are present in a weight ratio from about 0.05 to about 0.7.

28. The process of claim 2, wherein reducing said benzoquinone comprises:
contacting the benzoquinone with a reducing agent comprising hydrogen gas or hydrogen-containing gas mixtures in the presence of a reducing catalyst.

29. The process of claim 2, wherein the reducing agent is selected from the group consisting of sodium borohydride, sodium dithionate, lithium aluminum hydride, sodium hydro sulfite and sodium bisulfite.

30. The process of claim 2, further comprising isolating the hydroquinone compound comprises:
distilling the solvent; and
precipitating said hydroquinone compound using an anti-solvent.

31. The process of claim 30, wherein said anti-solvent is selected from the group consisting of toluene, xylene, and heptane.

32. A polycarbonate comprising structural units derived from the hydroquinone compound prepared in accordance with claim 2.

33. A polycarbonate prepared using the hydroquinone compound prepared in accordance with claim 2.

34. A process for preparing 2-methylhydroquinone, said process comprising:
oxidizing ortho-cresol with oxygen gas in a solvent selected from the group consisting of methylisobutylketone and methylethylketone in the presence of lithium trichlorocuprate dihydrate catalyst, and N-methylpyrrolidone promoter at a pH of about 1 to about 5 to form 2-methyl benzoquinone, wherein the lithium trichlorocuprate dihydrate catalyst is less than or equal to 0.1 mole per mole of the ortho-cresol;
reducing the 2-methyl benzoquinone; and
isolating the 2-methylhydroquinone.

35. A polycarbonate comprising structural units derived from the 2-methylhydroquinone prepared in accordance with claim 35.

36. A polycarbonate prepared using the 2-methylhydroquinone prepared in accordance with claim 35.

37. A process for preparing a benzoquinone compound, said process comprising:
oxidizing an aromatic hydroxy compound in a solvent with an oxygen gas or an oxygen-containing gas mixture in the presence of catalytic amount of a copper containing catalyst and a promoter to form the benzoquinone compound, wherein said promoter comprises an organic amide.

38. The process of claim 37, wherein the catalytic amount of the copper containing catalyst is less than or equal to 0.1 mole per mole of the ortho cresol.
